(19) United States
(12) Patent Application Publication
(10) Pub. No.: US 2015/0351728 A1
(43) Pub. Date: Dec. 10, 2015

(54) URINE SAMPLE DEVICE

(71) Applicant: Floyd Nathan STEWART, (US)

(72) Inventor: Floyd Nathan Stewart, Birmingham (GB)

(21) Appl. No.: 14/759,270

(22) PCT Filed: Jan. 9, 2014
(86) PCT No.: PCT/GB2014/050058

§ 371 Date: Jul. 6, 2015

(30) Foreign Application Priority Data
Jan. 9, 2013 (GB) 1300318.1
May 22, 2013 (GB) 1309242.4

(51) Int. Cl. A61B 10/00; A61G 9/00

(52) U.S. Cl. CPC A61B 10/007; A61G 9/006; A01K 23/00

(57) ABSTRACT

A device for collection of a mid-stream urine sample from a patient includes a catch device for the receipt of urine from a urine stream; a biasing component; and a release component; wherein the device has a first condition in which urine from a urine stream is unable to enter the catch device and a second condition in which urine from the urine stream is able to enter the catch device, said biasing component urging the apparatus towards the second condition and being operatively restrained by the release component; and wherein, with the device in the first condition, urine from a urine stream causes or is able to cause the release component to cease operatively restraining the biasing component whereby the device adopts the second condition under action of the biasing component.

The present invention relates to a device for collecting urine samples. In particular, it relates to a device that can readily collect a clean and uncontaminated urine sample, especially a clean and uncontaminated mid-stream urine sample. It also relates to a device that can be used to collect a urine sample from a person whilst they are carrying on their normal routine, and which can in particular be used for a child, an elderly person or a disabled person. The device can also be used for animals.

In the field of medicine, for both adults and children there is a need to obtain urine samples from time to time, as samples of urine may be used to test for various medical conditions. Samples of urine may be required on a one-off basis or may be required on a regular basis, e.g. for monitoring a condition.

In general, to obtain good results from tests on urine samples it is desirable that the sample is uncontaminated. Contamination may occur from contact of the urine with germs from another part of the body, e.g. the penis or vagina. Contamination may also occur from contact with germs or other contaminants in the urethra.

One current approach is to take a “mid-stream” sample. Thus the patient giving the urine sample should start to urinate but this initial part of the urine stream should not be directed into the sample tube. This initial part of the stream will contain any contaminants from the urethra. Shortly after the urine begins to flow, when the urethra will have been cleared of contaminants, the sample tube can be located in a position to begin capturing the urine. It will be used to collect a sample of about 1 to 2 ounces; this sample is generally known as the mid-stream sample.

However, such a procedure can be cumbersome, messy and unhygienic due to spills. It also requires that a judgement is made as to when to start collecting the mid-stream sample.

A “clean catch” procedure into a urine sample tube is also known. A “clean catch” sample is a urine specimen that is as free of bacterial contamination as possible without the use of a catheter. This type of specimen is needed to test urine for culture and sensitivity. After appropriate cleansing of the external genitalia, the client begins the urinary stream. As with the “mid-stream” approach, the initial portion is allowed to escape. The initial stream cleans or flushes the urethral orifice and meatus of resident bacteria. During the middle portion, the client collects the specimen. The urine sample tube should be held on the outside only and should not touch any other parts of the patient's body, especially the genital area, to avoid contamination from there.

Further, when using this technique to collect a urine sample for young children, especially babies and toddlers, the process is made more difficult due to the fact that young children cannot provide a urine sample on demand. Therefore a parent or carer has to wait for the patient to start to pass urine and then be ready to move the sample tube into position, which is very time-consuming. A similar problem can exist for the elderly or disabled, as well as for animals, e.g. pets.

In general, the task is a very frustrating, difficult and messy one and can be an unhygienic process due to spillages that can increase the risk of cross infection.

### SUMMARY OF THE INVENTION

The invention provides, in a first aspect, a device for collection of a mid-stream urine sample from a patient, the apparatus comprising:
a catch device for the receipt of urine from a urine stream;
a biasing component; and
a release component;

wherein the apparatus has a first condition in which urine from a urine stream is unable to enter the catch device and a second condition in which urine from the urine stream is able to enter the catch device, said biasing component urging the apparatus towards the second condition and being operatively restrained by the release component

and wherein, with the apparatus in the first condition, urine from a urine stream causes or is able to cause the release component to cease operatively restraining the biasing component whereby the apparatus adopts the second condition under action of the biasing component.

In the first configuration said biasing component may operatively urge the catch device into or towards an intended flow path of a urine stream. Thus it may be that in the first configuration the catch device is not aligned with the intended flow path of a urine stream. When the release component ceases operatively restraining the biasing component, the biasing component urges the catch device into or towards the intended flow path of a urine stream and the apparatus adopts the second condition. Thus in the second condition the catch device will be aligned with the intended flow path of a urine stream and can receive a urine sample.

In the first configuration, it may be that at least a portion of the urine stream may pool in, or flow to, the proximity of a break portion in the release component. The break portion may be arranged to fracture, tear, or at least become weakened, on contact with urine. As the break portion fractures, tears or otherwise weakens, the force of the biasing component overcomes the operative restraining force exerted by the release component. Thus the release component ceases operatively restraining the biasing component, and the apparatus adopts the second condition in which the biasing component urges the catch device into or towards the intended flow path of a urine stream. Thus urine from the urine stream is able to enter the catch device.

The device may comprise a baffle or other urine deflector arranged to intercept an intended flow path of a urine stream. Thus in the first configuration the catch device may be aligned with the intended flow path of a urine stream, but the baffle or other urine deflector intercepts the intended flow path and deflects the flow of urine to ensure that urine does not enter the catch device.

In the first configuration said biasing component may operatively urge the baffle or other urine deflector out of engagement with an intended flow path of a urine stream and said release component may operatively restrain said biasing component. Thus when the release component ceases operatively restraining the biasing component, the biasing component operatively urges the baffle or other urine deflector out of engagement with the intended flow path of a urine stream and the apparatus adopts the second condition. Thus in the second condition the catch device will be aligned with the intended flow path of a urine stream, with the baffle or other urine deflector no longer intercepting the intended flow path, and so the catch device can receive a urine sample.

In the first configuration, it may be that at least a portion of the urine stream may pool in, or flow to, the proximity of a break portion in the release component. The break portion may be arranged to fracture, tear, or at least become weakened, on contact with urine. As the break portion fractures, tears or otherwise weakens, the force of the biasing component overcomes the operative restraining force exerted by the release component. This causes the baffle or other urine deflector to be urged out of engagement with the intended flow path of the urine stream and the apparatus adopts the second condition in which the catch device will be aligned with the intended flow path of a urine stream, with the baffle or other urine deflector no longer intercepting the intended flow path. Thus urine from the urine stream is able to enter the catch device.

In the device of the present invention, contact from urine in a urine stream may cause, or be able to cause, the release component to cease operatively restraining the biasing component.

Particularly preferred embodiments are envisaged where the release component comprises a break portion that is weakened (e.g. at least partly dissolved or disintegrated) by contact with urine. As the break portion fractures, tears or otherwise weakens, the force of the biasing component overcomes the operative restraining force exerted by the release component. This break portion may be formed from, for example a urine-dissolvable material;

for example the portion may comprise one or more of (a) cellulose-based material, such as paper or card, or (b) a polymer that dissolves or disintegrates in the presence of water, or (c) a polymer that dissolves or disintegrates at the pH of urine, or (d) material that includes a pre-weakened section, e.g. a perforated section, that will break under the downward force of urine flowing onto it.

The device may comprise an initial catch device, said initial catch device preferably being arranged to collect urine from a urine stream with said apparatus in said first condition.

Therefore, the first aspect of the invention provides a device which, after a period of time exposed to a urine stream adopts a condition, under the action of a biasing component, whereby a mid-stream portion of the urine stream can be captured. The initial portion may be, for example, diverted, away from the catch device or, for example, the catch device may be moved to intercept the urine stream. In either case, if the device is used in combination with a nappy or other absorbent material, the initial portion of the stream may simply flow to waste, for example it can be soaked up by a nappy or other absorbent material or, alternatively, it can be captured by a further catch device (i.e. by an initial catch device).

A further or more specific aspect of the invention provides a urine collection device suitable for the collection of a mid-stream urine sample from a patient, the device comprising:
a first catch funnel for receipt of urine, the first catch funnel having an entry portion for receiving a urine stream and an exit portion for transferring said urine stream to a first urine collection chamber;
a second catch funnel for receipt of urine, the second catch funnel having an entry portion for receiving a urine stream and an exit portion for transferring said urine stream to a second urine collection chamber;
a support component on which the first catch funnel and second catch funnel are mounted;
a biasing component that biases the support component towards a position where the second catch funnel is located to receive the urine stream from the patient; and
a release component that releasably secures the support component against the bias of the biasing component, to be in a position where the first catch funnel is located to receive the urine stream;
   wherein the urine collection device can be initially provided in a first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to a first urine collection chamber, and wherein the urine collection device is able to subsequently be provided in a second configuration, in which the release component no longer secures the support component against the bias of the biasing component, such that the biasing component acts to move the support component to a position where the second catch funnel is located to receive the urine stream from the patient, and in this second configuration a mid-stream sample of the urine stream can be received in the entry portion of the second catch funnel and transferred via the exit portion of the second catch funnel to a second urine collection chamber.

Accordingly, this device of a further or more specific aspect of the invention may be seen as being in accordance with the first aspect, and as comprising:
as an or the initial catch device, a first catch funnel for receipt of urine, the first catch funnel having an entry portion for receiving a urine stream and an exit portion for transferring said urine stream to a first urine collection chamber;
as the catch device, a second catch funnel for receipt of urine, the second catch funnel having an entry portion for receiving a urine stream and an exit portion for transferring said urine stream to a second urine collection chamber;
a support component on which the first catch funnel and second catch funnel are mounted;

wherein the biasing component biases the support component towards a position where the second catch funnel is located to receive the urine stream from the patient; and

wherein the release component releasably secures the support component against the bias of the biasing component, to be in a position where the first catch funnel is located to receive the urine stream;

wherein the urine collection device can be initially provided in the first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to a first urine collection chamber,

and wherein the urine collection device is able to subsequently be provided in the second configuration, in which the release component no longer secures the support component against the bias of the biasing component, such that the biasing component acts to move the support component to a position where the second catch funnel is located to receive the urine stream from the patient, and in this second configuration a mid-stream sample of the urine stream can be received in the entry portion of the second catch funnel and transferred via the exit portion of the second catch funnel to a second urine collection chamber.

Thus hereinafter in the present application, descriptions of features in respect of the first catch funnel for receipt of urine will be understood to apply more generally to an or the initial catch device, as appropriate. Equally, in the present application, descriptions of features in respect of the second catch funnel for receipt of urine will be understood to apply more generally to the catch device.

The urine collection device of the further or more specific aspect therefore permits a first sample of urine to be collected in a first urine collection chamber; this first sample will be potentially contaminated with any contaminants that were present in the urethra. This first sample can be disposed of appropriately. The urine collection device then permits a second sample of urine to be collected in a second urine collection chamber; this second sample is the mid-stream sample that is desired for testing because it will be free of contaminants that were present in the urethra. The device is clearly advantageous in that it reduces mess and makes the process of collecting the desired mid-stream urine sample easy and straightforward. The collection device moves from collecting the first sample to collecting the desired mid-stream sample without the user needing to manually move a sample tube into position.

The urine collection device of the first or further or more specific aspect can be used to collect urine samples from adults or from children (including the elderly, disabled adults or children, babies or toddlers, adults or children who are sedated or in a coma) or from animals, especially pets (such as cats, dogs or horses). It can be hand held or it can be incorporated into an item of clothing, for example a nappy, underpants or an incontinence clothing article.

In one embodiment of the first aspect, the device is suitable for the collection of a urine sample from a patient whilst the patient is wearing a garment.

In one such embodiment, the device further comprises:
a first urine collection chamber for receipt of urine from the exit portion of the first catch funnel;
a first securing component for securing the first urine collection chamber to the patient's garment;
a second urine collection chamber for receipt of urine from the exit portion of the second catch funnel; and
a second securing component for securing the second urine collection chamber to the patient's garment;
   wherein the urine collection device is able to be provided in a configuration in which the first securing component secures the first urine collection chamber to the patient's garment, and the second securing component secures the second urine collection chamber to the patient's garment.

It may be that the urine collection device of the first aspect is provided in the form of a garment-based device that can be worn by the patient over a period of time.

In one such embodiment, the device further comprises:
a garment suitable for being worn by the patient around their lower torso and/or genital area;

wherein when the garment is worn by the patient around their lower torso and/or genital area, the first and second catch funnels are located inside the garment, adjacent to the patient's body. In the first configuration the entry portion of the first catch funnel will be located proximate to the patient's urethra, such that a sample of the patient's urine stream can be received in the entry portion of the first catch funnel and transferred via the exit portion of the first catch funnel to the first urine collection chamber. In the second configuration the entry portion of the second catch funnel will be located proximate the patient's urethra, such that a sample of the patient's urine stream can be received in the entry portion of the second catch funnel and transferred via the exit portion of the second catch funnel to the second urine collection chamber.

The present invention also provides, in a second aspect, a urine collection device suitable for the collection of a urine sample from a patient whilst the patient is wearing a garment, the device comprising:
a urine collection chamber;
a securing component for securing the urine collection chamber to the patient's garment; and
a catch funnel for receipt of urine, the catch funnel having an entry portion for receiving a urine stream from the patient and an exit portion for transferring said urine stream to the urine collection chamber;
   wherein the urine collection device is able to be provided in a configuration in which the securing component secures the urine collection chamber to the patient's garment, and the entry portion of the catch funnel is located proximate the patient's urethra, such that in this configuration a sample of the patient's urine stream can be received in the entry portion of the catch funnel and transferred via the exit portion of the catch funnel to the urine collection chamber.

The urine collection device of the second aspect therefore can be secured to the patient's garment, e.g. a nappy, underwear or an incontinence clothing article, and can be worn by the patient over a period of time. The patient therefore does not need to provide a urine sample on demand, or communicate to another person that they are ready to urinate, but instead will urinate when they are next ready to do so. When they do urinate, the proximity of the entry portion of the catch funnel to the patient's urethra means that the patient's urine stream is directly received in the entry portion of the catch funnel and is transferred via the exit portion of the catch funnel to the urine collection chamber.

The device is clearly advantageous in that there is no need for any part of the device that receives the urine to actually contact the patient's urethra or genital area, therefore reducing the risk of contamination. The device is secured to the patient's garment and the entry portion of the funnel does not need to contact the patient. Significantly, the collection device is secured to the patient's garment and the patient is able to freely move around and carry on their normal activities whilst wearing the device. This means that the patient can simply urinate when they are next ready to do so and the urine will be collected without the patient needing to do anything.

The urine collection device of the second aspect can be used to collect urine samples from adults or from children (including the elderly, disabled adults or children, babies or toddlers, adults or children who are sedated or in a coma) or from animals, especially pets (such as cats, dogs or horses). It is, however, particularly beneficial for the elderly, the disabled, those who are sedated or in a coma, babies and toddlers, due to the fact that it avoids the need for the patient to urinate on demand or to communicate that they are ready to urinate. For those same reasons it can also be useful for use with animals.

Although the garment to which the device will be attached will normally be a nappy, underpants or an incontinence clothing article or the like, it will be appreciated that the invention is not limited to being attached to any particular garment, as long as the garment is worn on the patient in a sufficiently secure fashion that it can bear the weight of the urine collection device, including when a urine sample has been provided into the urine collection chamber.

In one embodiment, the urine collection device of the second aspect is arranged to be suitable for the collection of a mid-stream urine sample from a patient.

Therefore in one such embodiment the device comprises
a first urine collection chamber;
a securing component for securing the first urine collection chamber to the patient's garment; and
a first catch funnel for receipt of urine, the catch funnel having an entry portion for receiving a urine stream from the patient and an exit portion for transferring said urine stream to the first urine collection chamber;
   wherein the urine collection device is able to be provided in a configuration in which the securing component secures the first urine collection chamber to the patient's garment, and the entry portion of the catch funnel is located proximate the patient's urethra, such that in this configuration a sample of the patient's urine stream can be received in the entry portion of the catch funnel and transferred via the exit portion of the catch funnel to the first urine collection chamber,
   and wherein the device further comprises:
a second urine collection chamber;
a securing component for securing the second urine collection chamber to the patient's garment;
a second catch funnel for receipt of urine, the second catch funnel having an entry portion for receiving a urine stream and an exit portion for transferring said urine stream to a second urine collection chamber;
   wherein the urine collection device is able to be provided in a configuration in which the securing component secures the second urine collection chamber to the patient's garment, and the entry portion of the second catch funnel is located proximate the patient's urethra, such that in this configuration a sample of the patient's urine stream can be received in the entry portion of the catch funnel and transferred via the exit portion of the catch funnel to the first urine collection chamber,
   and wherein the device further comprises:
a support component on which the first catch funnel and second catch funnel are mounted;
a biasing component that biases the support component towards a position where the second catch funnel is located to receive the urine stream from the patient; and
a release component that releasably secures the support component against the bias of the biasing component, to be in a position where the first catch funnel is located to receive the urine stream;
   wherein the urine collection device can be initially provided in a first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to the first urine collection chamber, and wherein the urine collection device is able to subsequently be provided in a second configuration, in which the release component no longer secures the support component against the bias of the biasing component, such that the biasing component acts to move the support component to a position where the second catch funnel is located to receive the urine stream from the patient, and in this second configuration a mid-stream sample of the urine stream can be received in the entry portion of the second catch funnel and transferred via the exit portion of the second catch funnel to the second urine collection chamber.

The present invention also provides, in a third aspect, a urine collection device suitable for the collection of a urine sample from a patient, the device comprising:
a garment suitable for being worn by the patient around their lower torso and/or genital area;
a urine collection chamber;
a catch funnel for receipt of urine, the catch funnel having an entry portion for receiving a urine stream from the patient and an exit portion for transferring said urine stream to the urine collection chamber;
   wherein when the garment is worn by the patient around their lower torso and/or genital area, the urine collection chamber is secured to the garment and the catch funnel is located inside the garment, adjacent to the patient's body, with the entry portion of the catch funnel being located proximate the patient's urethra, such that the urine collection device is in a configuration in which a sample of the patient's urine stream can be received in the entry portion of the catch funnel and transferred via the exit portion of the catch funnel to the urine collection chamber.

The urine collection device of the third aspect therefore can be worn by the patient over a period of time. The patient therefore does not need to provide a urine sample on demand, or communicate to another person that they are ready to urinate, but instead will urinate when they are next ready to do so. When they do urinate, the proximity of the entry portion of the catch funnel to the patient's urethra means that the patient's urine stream is directly received in the entry portion of the catch funnel and is transferred via the exit portion of the catch funnel to the urine collection chamber.

The device is clearly advantageous in that it allows a urine sample to be obtained that has less chance of being contaminated, because there is no need for any part of the catch funnel to actually contact the patient's urethra or genital area. The catch funnel is located within the garment and the urine collection chamber is secured to the garment and therefore, significantly, the patient is able to freely move around and carry on their normal activities whilst wearing the device. This means that the patient can simply urinate when they are next ready to do so and the urine will be collected without the patient needing to do anything.

The urine collection device of the third aspect can be used to collect urine samples from adults or from children (including the elderly, disabled adults or children, babies or toddlers, adults or children who are sedated or in a coma) or from animals, especially pets (such as cats, dogs or horses). It is, however, particularly beneficial for the elderly, the disabled, those who are sedated or in a coma, babies and toddlers, due to the fact that it avoids the need for the patient to urinate on demand or to communicate that they are ready to urinate. For those same reasons it can also be useful for use with animals.

Although the garment that is part of the device of the third aspect will normally be a nappy, underpants or an incontinence clothing article or the like, it will be appreciated that the invention is not limited to the use of any particular garment, as long as the garment is able to be worn by the patient around their lower torso and/or genital area in a sufficiently secure fashion that it can bear the weight of the catch funnel and urine collection chamber, including when a urine sample has been provided into the urine collection chamber.

In one embodiment, the urine collection device of the third aspect is arranged to be suitable for the collection of a mid-stream urine sample from a patient.

Therefore in one such embodiment the device comprises
a garment suitable for being worn by the patient around their lower torso and/or genital area;
a first urine collection chamber;
a first catch funnel for receipt of urine, the first catch funnel having an entry portion for receiving a urine stream from the patient and an exit portion for transferring said urine stream to the first urine collection chamber;
   wherein when the garment is worn by the patient around their lower torso and/or genital area, the first urine collection chamber is secured to the garment and the first catch funnel is located inside the garment, adjacent to the patient's body, and with the device being able to be provided in a configuration with the entry portion of the first catch funnel being located proximate the patient's urethra, such that the urine collection device is in a configuration in which a sample of the patient's urine stream can be received in the entry portion of the first catch funnel and transferred via the exit portion of the first catch funnel to the first urine collection chamber;
   and wherein the device further comprises:
a second urine collection chamber;
a second catch funnel for receipt of urine, the second catch funnel having an entry portion for receiving a urine stream and an exit portion for transferring said urine stream to a second urine collection chamber;
   wherein when the garment is worn by the patient around their lower torso and/or genital area, the second urine collection chamber is secured to the garment and the second catch funnel is located inside the garment, adjacent to the patient's body, and with the device being able to be provided in a configuration with the entry portion of the second catch funnel being located proximate the patient's urethra, such that the urine collection device is in a configuration in which a sample of the patient's urine stream can be received in the entry portion of the second catch funnel and transferred via the exit portion of the second catch funnel to the second urine collection chamber
   and wherein the device further comprises:
a support component on which the first catch funnel and second catch funnel are mounted;
a biasing component that biases the support component towards a position where the second catch funnel is located to receive the urine stream from the patient; and
a release component that releasably secures the support component against the bias of the biasing component, to be in a position where the first catch funnel is located to receive the urine stream;
   wherein the urine collection device can be initially provided in a first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to the first urine collection chamber, and wherein the urine collection device is able to subsequently be provided in a second configuration, in which the release component no longer secures the support component against the bias of the biasing component, such that the biasing component acts to move the support component to a position where the second catch funnel is located to receive the urine stream from the patient, and in this second configuration a mid-stream sample of the urine stream can be received in the entry portion of the second catch funnel and transferred via the exit portion of the second catch funnel to the second urine collection chamber.

In general, it will be appreciated that the first aspect and the second aspect of the invention may therefore be used in combination, and that the first aspect and the third aspect of the invention may therefore be used in combination. This applies in relation to all essential features and also to all features described as optional, unless it is apparent that this is not possible.

### DETAILED DESCRIPTION OF THE INVENTION

The urine collection device of an aspect, or of aspects, of the invention is, or are, designed for the collection of a mid-stream urine sample from a patient.

The device may have a first catch funnel for receipt of urine; this has an entry portion for receiving a urine stream and an exit portion for transferring said urine stream to a first urine chamber. The first catch funnel preferably has a greater cross sectional area at its entry portion than at its exit portion, to aid the catch of the urine stream from the patient, with reduced chance of contamination due to contact with the genital area, and then to funnel it to the first urine chamber.

The device may have a first urine chamber; this may be provided as part of the urine collection device, or may be provided as a separate component. The first urine chamber is for receiving the first sample of the urine stream. This will potentially be contaminated.

Therefore in some embodiments the intention is that the first urine sample will simply be disposed of appropriately. The exact shape, size and design of the first urine chamber is, therefore, not particularly significant to the present invention, provided that it is large enough to hold the first urine sample and can securely hold that sample until it is disposed of.

However, in other embodiments it may be that there is a desire to test this sample as well and therefore it will be appreciated that the first urine chamber may have the features described below in relation to the second urine chamber, which is for receiving a sample that is to be tested.

It may be that the chamber is a reusable container (that can therefore be emptied out, washed/sterilised and re-used) or it may be that it is a disposable container that is disposed of after a single use. It may optionally be provided with a lid or zip or other closure mechanism that can be used to close the chamber after the first sample of urine is in the chamber.

It may be that the chamber is a standard urine collection tube and a connector is provided to connect the funnel to such a tube. The connector may, for example, be push fit, screw fit or bayonet fit.

It may be that the chamber is a plastic or rubber container. It may, in one embodiment, be made from any material known for use in relation to catheter urinary drainage bags, e.g.

vinyl or latex.

It may be that the chamber is transparent or includes a transparent window section, so that it can be seen whether there is a urine sample inside.

In one embodiment the chamber is smaller than a catheter urinary drainage bag. In one embodiment the chamber has a volume of 350 ml or less such as 300 ml or less, e.g. from 10 to 250 ml, or from 25 to 200 ml, or from 50 to 150 ml. However, it could also be envisaged that the chamber could be larger than this, e.g. it might be from 350 ml to 700 ml in volume or more.

As the first urine chamber is only required to hold and receive the first (potentially contaminated) sample, it could suitably be that the chamber has a volume of from 20 to 100 ml, or from 30 to 90 ml, or from 50 to 75 ml.

The device may have a second catch funnel for receipt of urine; this has an entry portion for receiving a urine stream and an exit portion for transferring said urine stream to a second urine chamber. The second catch funnel preferably has a greater cross sectional area at its entry portion than at its exit portion, to aid the catch of the urine stream from the patient, with reduced chance of contamination due to contact with the genital area, and then to funnel it to the second urine chamber.

It will be appreciated that the first and second catch funnels do not need to be identical and in some embodiments they will be different from each other.

The second urine chamber may be provided as part of the urine collection device, or it may be provided as a separate component. The second urine chamber is for receiving the mid-stream sample of the urine stream, which is the sample to be retained and tested. The exact shape, size and design of the second urine chamber may be varied to some degree, but clearly it is important that this chamber is clean and sterile on receipt of the mid-stream sample and is able to securely hold and transfer the sample to a desired location, as well as being large enough to hold the mid-stream urine sample.

It may be that the second urine chamber is a reusable container (that can therefore be emptied out, washed/sterilised and re-used) or it may be that it is a disposable container that is disposed of after a single use. It may suitably be provided with a lid or zip or other closure mechanism that can be used to close the chamber after the mid-stream sample of urine is in the chamber. Preferably the second chamber is a plastic container.

It may be that the chamber is a standard urine collection tube and a connector is provided to connect the funnel to such a tube. The connector may, for example, be push fit, screw fit or bayonet fit.

It may be that the second urine chamber is shaped to have a smaller cross sectional area at its distal end (i.e. the closed end at which the urine sample will accumulate), such that as urine enters the chamber it is funnelled towards that smaller end. Thus, the urine chamber may include at least a portion that is funnel shaped (e.g. this may be the shape of a cone, or a truncated cone, or a triangular prism, or a truncated triangular prism, or the like). This small cross sectional area towards the distal end means that a small volume of urine will provide a greater depth (height) of fluid in the chamber, which is therefore easier to see. In contrast, if the chamber has a consistent cross section then the urine could pool or puddle at the bottom of the chamber and would be more difficult to see, at least until a larger urine sample had been provided.

Of course, it will be appreciated that the chamber need not have its maximum cross section at the proximal end—so the chamber might, for example, taper towards the proximal end as well as tapering towards the distal end. However, it is advantageous that the chamber does not have a consistent cross section from its proximal end to its distal end, and that the cross section at the distal end is less than the maximum cross section of the chamber.

Preferably the shape of the chamber is such that there is a tapered section extending from the part of the chamber that has the maximum cross section to the distal end of the chamber. In one embodiment, the distal end of the chamber presents the part of the chamber with the smallest cross section. In one embodiment, the part of the chamber that has the maximum cross section is at or near the proximal end. In one embodiment, the part of the chamber that has the maximum cross section is located nearer to the proximal end than the distal end.

In one embodiment the chamber includes a conical or frustoconical section or any other portion that includes walls that are angled inwardly to serve to funnel the urine towards the distal end.

The chamber may optionally be provided with one or more channels located inside the chamber that assist with channelling the urine towards the distal end. These may, for example, be straw like passages that may be linear or curvilinear and provide a channel from a location at or near the proximal end to channel the urine towards the distal end.

It may be that the second urine chamber is a plastic or rubber container. It may, in one embodiment, be made from any material known for use in relation to catheter urinary drainage bags, e.g. vinyl or latex.

It may be that the chamber is transparent or includes a transparent window section, so that it can be seen whether there is a urine sample inside.

In one embodiment the chamber is smaller than a catheter urinary drainage bag. In one embodiment the chamber has a volume of 350 ml or less such as 300 ml or less, e.g. from 10 to 250 ml, or from 25 to 200 ml, or from 50 to 150 ml. However, it could also be envisaged that the chamber could be larger than this, e.g. it might be from 350m1 to 700 ml in volume or more.

In the event that the second urine chamber is intended to hold and receive only the mid-stream sample, which is about one to two fluid ounces, it could suitably be that the chamber has a volume of from 30 to 100 ml, or from 50 to 90 ml, or from 60 to 75 ml. In the event that the second urine chamber is intended to hold and receive both the mid-stream sample, and also any further urine flow from the patient, however, it could be larger than this, e.g. from 75 to 700 ml, or from 100 to 600 ml, or from 150 to 500 ml.

Optionally, the second urine chamber contains a urine test strip, so that a test is carried out on the desired mid-stream sample as soon as it enters the chamber.

Optionally, the second urine chamber contains a trigger for an alarm, so that when urine enters the chamber an alarm provides an alert that the patient has urinated. The alarm may be audible, visual, or vibratory, or a combination thereof.

Optionally, the second urine chamber is provided with one or more air release valves, to assist the flow of urine.

Optionally, the second urine chamber is provided with a bar code or other markings or coded data. This can allow information about the chamber, the patient, the date of use and/or the place of use to be provided, e.g. a reference code to allow tracking of the chamber, the name/date of birth of the patient, or the name of the hospital or other medical or veterinary facility where the chamber is being used.

Optionally, the second urine chamber is provided with a label section on the outer surface on which information can presented, using a writing instrument, such as a pen, pencil, crayon or the like. This can allow information about the chamber, the patient, the date of use and/or the place of use to be provided, e.g. a reference code to allow tracking of the chamber, the name/date of birth of the patient, or the name of the hospital or other medical or veterinary facility where the chamber is being used.

Optionally, the second urine chamber contains a preserving agent to preserve the urine, e.g. boric acid.

It will be appreciated that the first and second urine chambers do not need to be identical and in some embodiments they will be different from each other.

The device has a support component on which the first catch funnel and second catch funnel are mounted. This component must be movable between a first position where the first catch funnel is located to receive the urine stream from the patient and a second position where the second catch funnel is located to receive the urine stream from the patient. Preferably the support component slides between these two positions. It is envisaged that the movement will be linear but potentially designs could be contemplated where the movement could be arcuate.

The device also has a biasing component that acts to bias the support component towards a position where the second catch funnel is located to receive the urine stream from the patient. The biasing component may suitably be a resilient or elasticated component, such as a spring, e.g. a helical spring. However, any component that serves the purpose of providing a biasing force towards a position where the second catch funnel is located to receive the urine stream from the patient can be used.

The device also includes a release component that releasably secures the support component against the bias of the biasing component, to be in a position where the first catch funnel is located to receive the urine stream.

The release component may be indirectly attached to the support component, for example via the first catch funnel, or may be directly attached to the support component. It will be appreciated that the release component could take many different forms provided that it is capable of securing the support component in a first position, against the bias of the biasing component, and of subsequently releasing the support component, such that the bias of the biasing component serves to move the support component to a second position.

Particularly preferred embodiments are envisaged where the release component comprises a break portion that is weakened (e.g. at least partly dissolved or disintegrated) by contact with urine, and wherein in the first configuration of the device this break portion is located such that it will be contacted by the urine stream as the urine passes from the patient to the first urine chamber, meaning that as the first sample of urine is collected the break portion becomes weaker and no longer has sufficient strength to act against the biasing component. The support component will therefore then be biased to the second position by the biasing component. In one embodiment just the contact of the urine stream on the break portion causes the break portion to weaken and break, thereby releasing the support component. In another embodiment, the contact of the urine stream on the break portion causes the break portion to weaken and the combination of this weakening and the biasing force from the biasing component that causes the break portion to break, thereby releasing the support component.

It may be that the break portion is made from a cellulose based material, such as paper or card, or from a polymer that dissolves or disintegrates in the presence of water, or from a polymer that dissolves or disintegrates at the pH of urine, or includes a pre-weakened section, e.g. a perforated section, that will break under the downward force of urine flowing onto it.

These preferred embodiments are beneficial in that the device will move from the first configuration to the second configuration automatically, due to the flow of urine over the break portion. The skilled reader will appreciate that the exact nature of the break portion can be designed to ensure that the device moves to the second configuration after a suitable amount of urine has been collected as the first sample, such that the mid-stream urine sample is free of contamination.

Accordingly, the urine collection device is initially provided in a first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to a first urine chamber.

Then the urine collection device moves to its second configuration, in which the release component no longer secures the support component against the bias of the biasing component, such that the biasing component serves to move the support component to a position where the second catch funnel is located to receive the urine stream from the patient, and in this second configuration a mid-stream sample of the urine stream can be received in the entry portion of the second catch funnel and transferred via the exit portion of the second catch funnel to a second urine chamber.

The urine collection device therefore permits a first sample of urine to be collected in a first urine chamber; this first sample will be potentially contaminated with any contaminants that were present in the urethra. This first sample can be disposed of appropriately. The urine collection device then permits a second sample of urine to be collected in a second urine chamber; this second sample is the mid-stream sample that is desired for testing because it will be free of contaminants that were present in the urethra. The collection technique of the invention moves from collecting the first sample to collecting the desired mid-stream sample without the user needing to manually move a sample tube into position.

It will be appreciated that the urine collection device is not limited to collecting two samples of urine. There could be more than two, e.g. three or four samples collected. In one embodiment, the urine collection device is designed to collect three samples of urine; it can therefore collect a first sample of urine (which will be potentially contaminated with any contaminants that were present in the urethra), a second sample of urine (which is the mid-stream sample that is desired for testing because it will be free of contaminants that were present in the urethra) and a third sample of urine (which is the remainder of the patient's urine flow after a suitable sized sample for testing has been taken).

In one embodiment, therefore, the device comprises:
a first catch funnel for receipt of urine, the first catch funnel having an entry portion for receiving a urine stream and an exit portion for transferring said urine stream to a first urine collection chamber;
a second catch funnel for receipt of urine, the second catch funnel having an entry portion for receiving a urine stream and an exit portion for transferring said urine stream to a second urine collection chamber;
a first support component on which the first catch funnel and second catch funnel are mounted;
a first biasing component that biases the first support component towards a position where the second catch funnel is located to receive the urine stream from the patient; and
a first release component that releasably secures the first support component against the bias of the first biasing component, to be in a position where the first catch funnel is located to receive the urine stream;
a third catch funnel for receipt of urine, the third catch funnel having an entry portion for receiving a urine stream and an exit portion for transferring said urine stream to a third urine collection chamber;
a second support component on which the second catch funnel and third catch funnel are mounted;
a second biasing component that biases the second support component towards a position where the third catch funnel is located to receive the urine stream from the patient; and
a second release component that releasably secures the second support component against the bias of the second biasing component, to be in a position where the second catch funnel is located to receive the urine stream;
   such that the urine collection device can be initially provided in a first configuration, in which the first release component secures the first support component against the bias of the first biasing component, with the first support component being in a position where the first catch funnel is located to receive the urine stream from the patient, and in this first configuration a first sample of the urine stream can be received in the entry portion of the first catch funnel and transferred via the exit portion of the first catch funnel to a first urine collection chamber, and wherein the urine collection device is able to subsequently be provided in a second configuration, in which the first release component no longer secures the first support component against the bias of the first biasing component, such that the first biasing component acts to move the first support component to a position where the second catch funnel is located to receive the urine stream from the patient, and in this second configuration a mid-stream sample of the urine stream can be received in the entry portion of the second catch funnel and transferred via the exit portion of the second catch funnel to a second urine collection chamber;
   and wherein the urine collection device is able to subsequently be provided in a third configuration, in which the second release component no longer secures the second support component against the bias of the second biasing component, such that the second biasing component acts to move the second support component to a position where the second catch funnel is located to receive the urine stream from the patient, and in this third configuration a third sample of the urine stream can be received in the entry portion of the third catch funnel and transferred via the exit portion of the third catch funnel to a third urine collection chamber.

In this embodiment, the third catch funnel may suitably be as described above in relation to the first and second catch funnels. However, it will be appreciated that the first, second and third catch funnels do not need to be identical and in some embodiments they will be different from each other.

Also, in this embodiment, the second support component may suitably be as described above in relation to the first support component. However, it will be appreciated that the first and second support components do not need to be identical and in some embodiments they will be different from each other.

Further, in this embodiment, the second biasing component may suitably be as described above in relation to the first biasing component. However, it will be appreciated that the first and second biasing components do not need to be identical and in some embodiments they will be different from each other.

Finally, in this embodiment, the second release component may suitably be as described above in relation to the first release component. However, it will be appreciated that the first and second release components do not need to be identical and in some embodiments they will be different from each

The third urine chamber may be provided as part of the urine collection device, or may be provided as a separate component. The third urine chamber is for receiving the third sample of the urine stream, which will be the patient's continued urine flow after the mid-stream sample for testing has been taken. Therefore the intention is that the third urine sample will simply be disposed of appropriately. The exact shape, size and design of the third urine chamber is, therefore, not particularly significant to the present invention, provided that it is large enough to hold the third urine sample and can securely hold that sample until it is disposed of.

It may be that the chamber is a reusable container (that can therefore be emptied out, washed/sterilised and re-used) or it may be that it is a disposable container that is disposed of after a single use. It may optionally be provided with a lid or zip or other closure mechanism that can be used to close the chamber after the third sample of urine is in the chamber.

It may be that the chamber is a plastic or rubber container. It may, in one embodiment, be made from any material known for use in relation to catheter urinary drainage bags, e.g. vinyl or latex.

It may be that the chamber is transparent or includes a transparent window section, so that it can be seen whether there is a urine sample inside. In one embodiment the third chamber is smaller than a catheter urinary drainage bag. In one embodiment the chamber has a volume of 350 ml or less, such as 300 ml or less, e.g. from 50 to 300 ml, or from 75 to 250 ml, or from 100 to 200 ml. However, it could also be envisaged that the chamber could be larger than this, e.g. it might be from 350 ml to 700 ml in volume or more.

The first and second (and the optional third) urine collection chambers may be located inside the garment when the garment is worn by the patient around their lower torso and/or genital area. Alternatively, they may be provided in a manner such that they are partially or fully external to the garment when the garment is worn, e.g. they may be attached to the outside of the garment.

It is preferred that the urine collection chambers are located inside the garment, as this reduces the chance of the user displacing or damaging the urine collection chambers. However, it will be appreciated that for some patients this is less of a concern than for others. So for an elderly patient or a sedated patient, for example, there is less of a concern about locating the urine collection chambers outside the garment than for a child or an animal.

When the urine collection chambers are located inside the garment, it may be that at least part of the second collection chamber is visible from outside the garment, so that it can be observed when there is urine in that chamber, which is of course the chamber that will receive the sample to be tested. For example, it may be that there is a hole or window provided in the garment in a location such that a portion of the second collection chamber can be seen through this hole or window. Clearly the portion of the second collection chamber can be seen through this hole or window should be a lower portion of the chamber (i.e. towards the closed end of the chamber), as this is where urine will accumulate first. The window may be formed from any suitable material that is sufficiently transparent for the presence of urine to be seen therethrough, e.g. it may be a plastic material that is transparent or translucent. Alternatively, a portion of the second collection chamber may protrude though a hole in the garment. Clearly the portion of the second collection chamber protruding through this hole should be a lower portion of the chamber (i.e. towards the closed end of the chamber), as this is where urine will accumulate first.

It will be appreciated that if the urine collection chambers are to be located outside the garment then the associated catch funnels must be connected thereto, either through the garment or round the garment. Thus there may be a hole in the garment through which the catch funnel and associated collection chamber are linked, (e.g. directly or via a connector, such as tubing), or the catch funnel and the associated collection chamber may be connected round the garment, e.g. over the top of the garment or under the bottom of the garment (this may, for example, be via a connector, such as flexible tubing).

The urine collection device of the second or other aspect of the invention is designed for the collection of a urine sample from a patient, whilst allowing the patient to carry on with their normal routine and activities.

The device includes a urine collection chamber; this is for receiving the sample of the urine stream from the patient. The urine collection chamber has an entry section for receiving the sample of the urine stream from the patient. This may be any suitable opening and may optionally be provided in a neck portion of the container.

The exact shape, size and design of the urine chamber may be varied to some degree, but clearly it is important that this chamber is clean and sterile on receipt of the urine sample and is able to securely hold and transfer the sample to a desired location, as well as being large enough to hold the urine sample.

It may be that the chamber is a reusable container (that can therefore be emptied out, washed/sterilised and re-used) or it may be that it is a disposable container that is disposed of after a single use.

It may suitably be shaped and sized such that it can be provided with a lid or zip or other closure mechanism that can be used to close the chamber after the sample of urine is in the chamber, so that the chamber containing the urine can then be stored and/or transported. However, in another embodiment it is envisaged that the urine sample would be directly transferred to a separate container for storage and/or transportation and therefore the urine collection chamber does not need to be shaped and sized such that it can be provided with a lid that can be used to close the chamber.

It may be that the chamber is a standard urine collection tube and a connector is provided to connect the funnel to such a tube. The connector may, for example, be push fit, screw fit or bayonet fit.

Preferably the chamber is a plastic container or a rubber container. It may, in one embodiment, be made from any material known for use in relation to catheter urinary drainage bags, e.g. vinyl or latex.

When the chamber is intended to be located within the patient's garment, it is preferred that the chamber is made from a flexible plastic and/or rubber material that can therefore flex into a position between the garment and the patient's body to increase the comfort of the patient when using the device.

However, it may be that the chamber is intended to be attached to the outside of the patient's garment. Whilst in that embodiment the chamber may be made from a flexible plastic and/or rubber material that can therefore flex into a position against the patient's body, to increase the comfort of the patient when using the device, it could also be envisaged in this embodiment that the chamber might be made from a rigid material, e.g. rigid plastic, that could form a box on the outside of the garment.

It may be that the chamber is transparent or includes a transparent window section, so that it can be seen whether there is a urine sample inside.

It may be that the urine chamber is shaped to have a smaller cross sectional area at its distal end (i.e. the closed end at which the urine sample will accumulate), such that as urine enters the chamber it is funnelled towards that smaller end. Thus, the urine chamber may include at least a portion that is funnel shaped (e.g. this may be the shape of a cone, or a truncated cone, or a triangular prism, or a truncated triangular prism, or the like). This small cross sectional area towards the distal end means that a small volume of urine will provide a greater depth (height) of fluid in the chamber, which is therefore easier to see. In contrast, if the chamber has a consistent cross section then the urine could pool or puddle at the bottom of the chamber and would be more difficult to see, at least until a larger urine sample had been provided.

Of course, it will be appreciated that the chamber need not have its maximum cross section at the proximal end—so the chamber might, for example, taper towards the proximal end as well as tapering towards the distal end. However, it is advantageous that the chamber does not have a consistent cross section from its proximal end to its distal end, and that the cross section at the distal end is less than the maximum cross section of the chamber.

Preferably the shape of the chamber is such that there is a tapered section extending from the part of the chamber that has the maximum cross section to the distal end of the chamber. In one embodiment, the distal end of the chamber presents the part of the chamber with the smallest cross section. In one embodiment, the part of the chamber that has the maximum cross section is at or near the proximal end. In one embodiment, the part of the chamber that has the maximum cross section is located nearer to the proximal end than the distal end.

In one embodiment the chamber includes a conical or frustoconical section or any other portion that includes walls that are angled inwardly to serve to funnel the urine towards the distal end.

The chamber may optionally be provided with one or more channels located inside the chamber that assist with channelling the urine towards the distal end. These may, for example, be straw like passages that may be linear or curvilinear and provide a channel from a location at or near the proximal end to channel the urine towards the distal end.

In one embodiment the chamber is smaller than a catheter urinary drainage bag. In one embodiment the chamber has a volume of 350 ml or less such as 300 ml or less, e.g. from 10 to 250 ml, or from 25 to 200 ml, or from 50 to 150 ml. However, it could also be envisaged that the chamber could be larger than this, e.g. it might be from 350 ml to 700 ml in volume or more.

Optionally, the urine chamber contains a urine test strip, so that a test is carried out on the urine sample as soon as it enters the chamber.

Optionally, the urine chamber contains a trigger for an alarm, so that when urine enters the chamber an alarm provides an alert that the patient has urinated. The alarm may be audible, visual, or vibratory, or a combination thereof.

Optionally, the urine chamber is provided with one or more air release valves, to assist the flow of urine.

Optionally, the urine chamber is provided with a bar code or other markings or coded data. This can allow information about the chamber, the patient, the date of use and/or the place of use to be provided, e.g. a reference code to allow tracking of the chamber, the name/date of birth of the patient, or the name of the hospital or other medical or veterinary facility where the chamber is being used.

Optionally, the urine chamber is provided with a label section on the outer surface on which information can presented, using a writing instrument, such as a pen, pencil, crayon or the like. This can allow information about the chamber, the patient, the date of use and/or the place of use to be provided, e.g. a reference code to allow tracking of the chamber, the name/date of birth of the patient, or the name of the hospital or other medical or veterinary facility where the chamber is being used.

Optionally, the urine chamber contains a preserving agent to preserve the urine, e.g. boric acid.

The device also includes a securing component for securing the urine collection chamber to the patient's garment.

The urine collection chamber may be secured inside the garment. Alternatively, it may be secured such that it is partially or fully external to the garment when the garment is worn, e.g. it may be secured to the outside of the garment.

It is preferred that the urine collection chamber is secured inside the garment, as this reduces the chance of the user displacing or damaging the urine collection chamber. However, it will be appreciated that for some patients this is less of a concern than for others. So for an elderly patient or a sedated patient, for example, there is less of a concern about securing the urine collection chamber outside the garment than for a child or an animal.

When the urine collection chamber is secured inside the garment, it may be that at least part of the collection chamber is visible from outside the garment, so that it can be observed when there is urine in that chamber. For example, it may be that there is a hole or window provided in the garment in a location such that a portion of the collection chamber can be seen through this hole or window. Clearly the portion of the collection chamber can be seen through this hole or window should be a lower portion of the chamber (i.e. towards the closed end of the chamber), as this is where urine will accumulate first. The window may be formed from any suitable material that is sufficiently transparent for the presence of urine to be seen therethrough, e.g. it may be a plastic material that is transparent or translucent. Alternatively, a portion of the collection chamber may protrude though a hole in the garment. Clearly the portion of the collection chamber protruding through this hole should be a lower portion of the chamber (i.e. towards the closed end of the chamber), as this is where urine will accumulate first.

It will be appreciated that if the urine collection chamber is to be secured outside the garment then the associated catch funnel must be connected thereto, either through the garment or round the garment. Thus there may be a hole in the garment through which the catch funnel and the collection chamber are linked, (e.g. directly or via a connector, such as tubing), or the catch funnel and the collection chamber may be connected round the garment, e.g. over the top of the garment or under the bottom of the garment (this may, for example, be via a connector, such as flexible tubing).

The securing component for securing the chamber to the garment (either on its interior or exterior) may involve mechanical securing devices or chemical adhesives. The mechanical securing devices may involve male and female connectors, e.g. buttons and related holes, snap fit connectors, push fit connectors, zips or the like. The chemical adhesives may be of the type known for use on sanitary pads and incontinence pads, which are used to releasably secure such pads to the inside of underpants, and may therefore serve in this invention to releasably secure the urine chamber to the patient's garment.

The securing component may involve the use of high friction material. The material may, for example, have a kinetic coefficient of friction of 0.3 or higher, 0.4 or higher, 0.5 or higher, 0.6 or higher, or 0.7 or higher. A kinetic coefficient of friction may be measured according to the ASTM D 1894 protocol.

High friction properties will grip the securing component to the fibres of the patient's garment and therefore the securing component provided with the high friction material can simply be pressed onto the patient's garment to secure the urine collection chamber to the inside or outside of the garment.

In one embodiment, the securing component may comprise a hook type securing device or hook and loop type securing device, such as that available under the Velcro® brand. In particular, the use of a hook type securing device or hook and loop type securing device with small hooks/loops (e.g. hooks/loops of the order of tens to hundreds of micrometres in width and height; such as from 10 to 100 micrometres in both width and height, or from 10 to 50 micrometres, in both width and height) may be contemplated, such as micro Velcro®.

The benefit of such a securing component is that the hooks of such a device will attach directly to the fibres of the patient's garment. Therefore the securing component tabs can simply be pressed onto the garment to secure the urine collection chamber to the inside or outside of the garment.

Therefore, as the reader will appreciate, when a hook and loop type securing device such as a Velcro® securing device is used, it is only the hook sections of such a device that are required to be used in the present invention, as the fabric of the patient's garment provides loop sections to which the hooks can attach.

In another embodiment, the securing component may comprise a flocked material. As the reader will appreciate, flocked material has many small fibre ‘flocks’ deposited on its surface. In particular, flocked material may have short monofilament fibres on its surface. The ‘flock’ fibres are upstanding on the surface, which gives good slip-or-grip friction properties.

The ‘flock’ fibres may comprise natural or synthetic fibres, for example the fibres may be nylon, rayon or polyester. The fibres may have a diameter of, for example, from 1×10⁻⁴m to 1×10⁻⁶m, in particular the order of about 10⁻⁵ m, e.g. from 0.5×10⁻⁵m to 5×10⁻⁵m. The fibres may have a length of, for example, from 0.1 to 10 mm, preferably from 0.25 to 5 mm. The fibres may be provided at a fibre density of, for example, 10 to 500 fibres per mm², e.g. from ‘flock’ fibres are provided may in one embodiment be a textile material but may also be paper or plastic.

Flocked material may be produced by the application of a high-voltage electric field to cause the application of the fibres to a surface of the material that has been coated with adhesive.

The flocked material may, for example, be of the type used in Fuzzy Felt®. Clearly, it is preferred that the material on which the ‘flock’ fibres are provided is, however, flexible rather than stiff.

The benefit of such a securing component is that the ‘flock’ fibres of the material will directly secure the pad to the fibres of the patient's garment, due to the slip-or-grip friction properties of the flocked material. Therefore the securing component tabs can simply be pressed onto the garment to secure the urine collection chamber to the inside or outside of the garment.

The securing component may be made up of a single section that secures the urine collection chamber to the patient's garment or may be made up of two or more sections that secure the urine collection chamber to the patient's garment. In one embodiment, there are two or more such sections, such as three or more, or four or more. In one embodiment there are at least two such sections and these two sections are located such that one is on the left side of the urine collection chamber and one is on the right side, or such that one is on the top side of the urine collection chamber and one is on the bottom side; most preferably they are symmetrically located so as to equally spread the load of the urine collection chamber.

In addition, the device includes a catch funnel for receipt of urine, the catch funnel having an entry portion for receiving a urine stream from the patient and an exit portion for transferring said urine stream to the urine collection chamber.

The catch funnel preferably has a greater cross sectional area at its entry portion than at its exit portion, to aid the catch of the urine stream from the patient, with reduced chance of contamination due to contact with the genital area, and then to funnel it to the urine collection chamber.

The catch funnel may link directly to the urine collection chamber. Alternatively, there may be one or more connectors between the catch funnel and the urine collection chamber. For example, the catch funnel may be provided with a funnel connector that links with the exit portion of the funnel and serves to transfer the urine stream to the urine collection chamber. Alternatively or additionally the urine collection chamber may be provided with a chamber connector that links with the entry section of the chamber and serves to receive the sample of the urine stream and transfer it to the chamber's entry section. Any such connectors may suitably be tubular. They may be plastic tubes, especially flexible plastic tubes.

Therefore the urine collection device of the second aspect is able to be provided in a configuration in which the securing component secures the urine collection chamber to the patient's garment, and the entry portion of the catch funnel is located proximate the patient's urethra, such that in this configuration a sample of the patient's urine stream can be received in the entry portion of the catch funnel and transferred via the exit portion of the catch funnel to the urine collection chamber.

The urine collection device of the second aspect therefore can be secured to the patient's garment, e.g. a nappy, underwear or an incontinence clothing article, and can be worn by the patient over a period of time. The patient therefore does not need to provide a urine sample on demand, or communicate to another person that they are ready to urinate, but instead will urinate when they are next ready to do so. When they do urinate, the proximity of the entry portion of the catch funnel to the patient's urethra means that the patient's urine stream is directly received in the entry portion of the catch funnel and is transferred via the exit portion of the catch funnel to the urine collection chamber.

The device is clearly advantageous in that it allows a urine sample to be obtained that has less chance of being contaminated, because there is no need for any part of the catch funnel to actually contact the patient's urethra or genital area. Significantly, the collection device is secured to the patient's garment and the patient is able to freely move around and carry on their normal activities whilst wearing the device. This means that the patient can simply urinate when they are next ready to do so and the urine will be collected without the patient needing to do anything.

The urine collection device of the third aspect includes a garment to be worn by the patient around their lower torso and/or genital area. The garment may suitably be a nappy, underpants or an incontinence clothing article. However, it could be a belt that can strap around the lower torso, or a pair of shorts or trunks, or any other similar garment. It will be appreciated that the invention is not limited to being any particular garment, as long as the garment is worn by the patient around their lower torso and/or genital area in a sufficiently secure fashion that it can bear the weight of the catch funnel and urine collection chamber, including when a urine sample has been provided into the urine collection chamber.

Preferably the chamber is a plastic container or a rubber container. It may, in one embodiment, be made from any material known for use in relation to catheter urinary drainage bags, e.g. vinyl and/or latex.

When the chamber is secured inside the patient's garment, it is preferred that the chamber is made from a flexible plastic and/or rubber material that can therefore flex into a position between the garment and the patient's body to increase the comfort of the patient when using the device.

However, it may be that the chamber is secured to the outside of the patient's garment. Whilst in that embodiment the chamber may be made from a flexible plastic and/or rubber material that can therefore flex into a position against the patient's body, to increase the comfort of the patient when using the device, it could also be envisaged in this embodiment that the chamber might be made from a rigid material, e.g. rigid plastic, that could form a box on the outside of the garment.

The urine collection chamber may be secured inside the garment, as this reduces the chance of the user displacing or damaging the urine collection chambers. However, it will be appreciated that for some patients this is less of a concern than for others. So for an elderly patient or a sedated patient, for example, there is less of a concern about locating the urine collection chamber outside the garment than for a child or an animal.

When the urine collection chamber is secured inside the garment, it may be that at least part of the collection chamber is visible from outside the garment, so that it can be observed when there is urine in that chamber, which is of course the chamber that will receive the sample to be tested. For example, it may be that there is a hole or window provided in the garment in a location such that a portion of the collection chamber can be seen through this hole or window. Clearly the portion of the collection chamber can be seen through this hole or window should be a lower portion of the chamber (i.e. towards the closed end of the chamber), as this is where urine will accumulate first. The window may be formed from any suitable material that is sufficiently transparent for the presence of urine to be seen therethrough, e.g. it may be a plastic material that is transparent or translucent. Alternatively, a portion of the collection chamber may protrude though a hole in the garment. Clearly the portion of the collection chamber protruding through this hole should be a lower portion of the chamber (i.e. towards the closed end of the chamber), as this is where urine will accumulate first.

The chamber may be permanently secured to the garment, e.g. it may be adhered thereto with adhesive, or it may be stitched to the garment, or it may be removably secured to the garment, e.g. using a securing component. The securing component may be as described above in relation to the second aspect.

In addition, the urine collection device preferably includes a catch funnel for receipt of urine. The catch funnel has an entry portion for receiving a urine stream from the patient and an exit portion for transferring said urine stream to the urine collection chamber.

The catch funnel preferably has a greater cross sectional area at its entry portion than at its exit portion, to aid the catch of the urine stream from the patient (with a reduced chance of it being contaminated, because there is no need for any part of the catch funnel to actually contact the patient's urethra or genital area) and then to funnel it to the urine collection chamber.

The catch funnel may link directly to the urine collection chamber. Alternatively, there may be one or more connectors between the catch funnel and the urine collection chamber. For example, the catch funnel may be provided with a funnel connector that links with the exit portion of the funnel and serves to transfer the urine stream to the urine collection chamber. Alternatively or additionally the urine collection chamber may be provided with a chamber connector that links with the entry section of the chamber and serves to receive the sample of the urine stream and transfer it to the chamber's entry section. Any such connectors may suitably be tubular. They may be plastic tubes, especially flexible plastic tubes.

It is therefore the case that when the garment is worn by the patient on their lower torso and/or genital area, the urine collection chamber and the catch funnel are located inside the garment, adjacent to the patient's body, with the entry portion of the catch funnel being located proximate the patient's urethra, such that the urine collection device is in a configuration in which a sample of the patient's urine stream can be received in the entry portion of the catch funnel and transferred via the exit portion of the catch funnel to the urine collection chamber.

The urine collection device of the third aspect therefore can be worn by the patient over a period of time. The patient therefore does not need to provide a urine sample on demand, or communicate to another person that they are ready to urinate, but instead will urinate when they are next ready to do so. When they do urinate, the proximity of the entry portion of the catch funnel to the patient's urethra means that the patient's urine stream is directly received in the entry portion of the catch funnel and is transferred via the exit portion of the catch funnel to the urine collection chamber.

### Urine Testing

In all aspects of the invention the urine may be tested manually or automatically.

It may be that the urine to be tested is collected in a urine collection chamber and that urine collection chamber is transferred to a location where a test is carried out.

It may be that the urine to be tested is collected in a urine collection chamber and then the urine to be tested is transferred to a test container in which a test is carried out.

It may be that, as mentioned above, the urine collection chamber includes a urine test strip, so that a test is carried out on the urine sample as soon as it enters the chamber. It may be that the chamber is transparent or includes a transparent window section, so that it can be seen whether there is a urine sample inside and so that the test results can be directly seen.

As is conventional, the urine test strip may comprise one or more (e.g. from two to ten) different chemical pads or reagents which may react following contact with a urine sample. The reaction suitably causes a change in colour. The urine test strip may, for example, test for diseases of the kidneys and the urinary tract; and/or carbohydrate metabolism disorders (diabetes mellitus); and/or liver diseases and haemolytic disorders.

It may be that the urine collection chamber includes an automated urine tester, which carries out a test on the urine sample as soon as it enters the chamber and records the result. It may be that the automated urine tester records the results of the tests. The automated urine tester may be removed from the urine collection chamber to note the results. Alternatively, the automated urine tester may transmit the results to a receiving device, e.g. by wireless communication such as radio communication, microwave communication, infrared communication, or ultrasonic communication. In one embodiment, short-wavelength radio transmissions are used, e.g. via Bluetooth.

It may be that the urine collection chamber includes a sampling device which absorbs or contains a sample of urine from within the chamber. This sampling device can then be removed and taken for testing.

In one embodiment there may be a machine provided that runs one or more tests on the urine sample.

### SPECIFIC DESCRIPTION WITH REFERENCE TO THE DRAWINGS

The invention will now be further described, in a non limiting manner, with reference to the drawings, in which:

FIG. 1 is a perspective view of a urine collection device of the invention; this is suitable for the collection of a mid-stream urine sample from a patient and is designed for the collection of a “clean-catch” urine sample from a patient, whilst allowing the patient to carry on with their normal routine and activities;

FIG. 2 is a plan view of a garment that can be used with a urine collection device of the invention;

FIG. 3 is a cross sectional view showing detail of the urine collection device of FIG. 1;

FIG. 4 is a cross sectional view showing detail of the urine collection device of FIG. 1 in operation, when the first urine sample is being collected;

FIG. 5 is a cross sectional view showing detail of the urine collection device of FIG. 1 in operation, when the second, mid-stream, urine sample is being collected;

FIG. 6 is a side view of the urine collection device of FIG. 1;

FIG. 7 is a perspective view showing detail of the urine collection device of FIG. 1;

FIG. 8 is an end view showing detail of the urine collection device of FIG. 1;

FIG. 9 is a perspective view of another urine collection device of the invention; this is designed for the collection of a “clean-catch” urine sample from a patient, whilst allowing the patient to carry on with their normal routine and activities

FIG. 10 is a side view showing detail of the urine collection device of FIG. 9;

FIG. 11 is a view from above showing detail of the urine collection device of FIG. 9;

FIG. 12 is a perspective view showing detail of the urine collection device of FIG. 9.

FIG. 13 is a cross section of another urine collection device of the invention; this is suitable for the collection of a mid-stream urine sample from a patient and is designed for the collection of a “clean-catch” urine sample from a patient, whilst allowing the patient to carry on with their normal routine and activities.

FIG. 14 is a cross section of another urine collection device of the invention; this is suitable for the collection of a mid-stream urine sample from a patient and is designed for the collection of a “clean-catch” urine sample from a patient, whilst allowing the patient to carry on with their normal routine and activities.

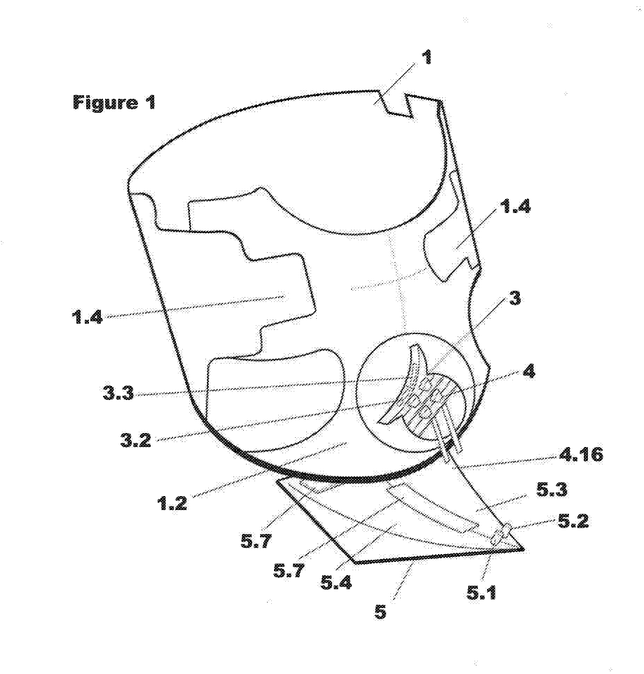

FIGS. 1-8 show a urine collection device of the present invention. This device is designed for the collection of a mid-stream urine sample from a patient. This device is also designed for the collection of a “clean-catch” urine sample from a patient, whilst allowing the patient to carry on with their normal routine and activities.

The device 3 has a first catch funnel 4.1 for receipt of urine; this has an entry portion for receiving a urine stream and an exit portion for transferring said urine stream to a first urine chamber 5.2. The first catch funnel suitably has a greater cross sectional area at its entry portion than at its exit portion, to aid the catch of the urine stream from the patient (with a reduced risk of contamination because there is no need for any part of the catch funnel to actually contact the patient's urethra or genital area) and then to funnel it to the first urine chamber. The first urine chamber is for receiving the first sample of the urine stream, which will potentially be contaminated.

The device also has a second catch funnel 4.2 for receipt of urine; this has an entry portion for receiving a urine stream and an exit portion for transferring said urine stream to a second urine chamber 5.1. The second catch funnel suitably has a greater cross sectional area at its entry portion than at its exit portion, to aid the catch of the urine stream from the patient (with a reduced risk of contamination because there is no need for any part of the catch funnel to actually contact the patient's urethra or genital area) and then to funnel it to the second urine chamber. The second urine chamber is for receiving the mid-stream sample of the urine stream, which is the sample to be retained and tested.

The device also has a support component 4.7 on which the first catch funnel and second catch funnel are mounted. This component is slidable in a linear fashion between a first position where the first catch funnel is located to receive the urine stream from the patient and a second position where the second catch funnel is located to receive the urine stream from the patient.

The device also has a biasing component 4.8 that acts to bias the support component towards a position where the second catch funnel is located to receive the urine stream from the patient. The biasing component is a helical spring or the like. In general, the biasing component may be a spring or, for example a wish bone, or other urging member.

The device also includes a release component 4.9 that releasably secures the support component against the bias of the biasing component, to be in a position where the first catch funnel is located to receive the urine stream.

The release component comprises a break portion that is weakened (e.g. at least partly dissolved or disintegrated) by contact with urine. In this regard, the break portion may fracture, tear or otherwise be weakened. In the first configuration of the device this break portion is located such that it will be contacted by the urine stream as the urine passes from the patient to the first urine chamber, meaning that as the first sample of urine is collected the break portion becomes weaker and no longer has sufficient strength to act against the biasing component. The support component will therefore then be biased to the second position by the biasing component.

The break portion may be made from a cellulose based material, such as paper or card, or from a polymer that dissolves or disintegrates in the presence of water, or from a polymer that dissolves or disintegrates at the pH of urine, or may include a pre-weakened section, e.g. a perforated section, that will break under the downward force of urine flowing onto it.

The inherent strength of the break portion is chosen according to the intended user (e.g. taking into account the velocity, volume, or other characteristic of the intended urine stream). For example, for an infant (baby or toddler) the strength may be low; for a large animal (e.g. a horse) the strength may be relatively high. Moreover, if it is required that a relatively large sample is taken the strength of the break portion may be chosen such that it weakens only when it has been exposed to a relatively large amount of urine, for example. Weakening of the break portion may be carried out in any of the ways contemplated above.

Accordingly, the urine collection device is initially provided in a first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to the first urine chamber.

Then the urine breaks the release component and the urine collection device moves to its second configuration, in which the release component no longer secures the support component against the bias of the biasing component, such that the biasing component serves to move the support component to a position where the second catch funnel is located to receive the urine stream from the patient, and in this second configuration a mid-stream sample of the urine stream can be received in the entry portion of the second catch funnel and transferred via the exit portion of the second catch funnel to the second urine chamber.

FIGS. 1-8 also show the article of clothing 1 to which the device can be secured. The article of clothing may be a nappy or the like which has fastening fixings 1.4 and a foundation area 1.2. There is an opening 1.1 leading between sheets and an opening 1.3 for connectors.

The urine collection device includes a flexible support 3.2 and an opening to the device 3.3.

The portion 4 of the device that allows the catch of the urine sample includes the catch funnels 4.1 and 4.2, the urine chambers 4.3 and 4.4, absorbent material 4.5 and 4.6 to soak up any urine spills or overflow, support component 4.7, biasing component 4.8, release component 4.9, flow valves 4.10 and 4.11, an encompassed surface 4.12 (with funnels), mountings 4.13 and 4.14 for the biasing component, fixture 4.15 for the release component, connectors 4.16 (to connect to the urine chambers) and perforations 4.18.

The urine stream is marked as 4.17.

The urine collection bag 5 has connectors 5.1 and 5.2, transfer areas 5.3 and 5.4, air release valves 5.5, Diagnostic strip 5.6, fixings 5.7 and check valves 5.8.

Considering each drawing in turn, the following description can be provided:

In FIG. 1, a urine collection apparatus includes the article of clothing 1 comprising fastening fixings 1.4 to tighten around the wearer waist; and foundation area 1.2 to fit the urine collection device 3 inside. The collection device 3 comprises a ‘catch’ apparatus 4 for collecting the first and mid-stream urine sample. The lower end of the article of clothing is fitted with urine collection bag 5 to store the urine samples; the urine collection bag is fitted with fixings 5.7 to mount onto the urine collection apparatus.

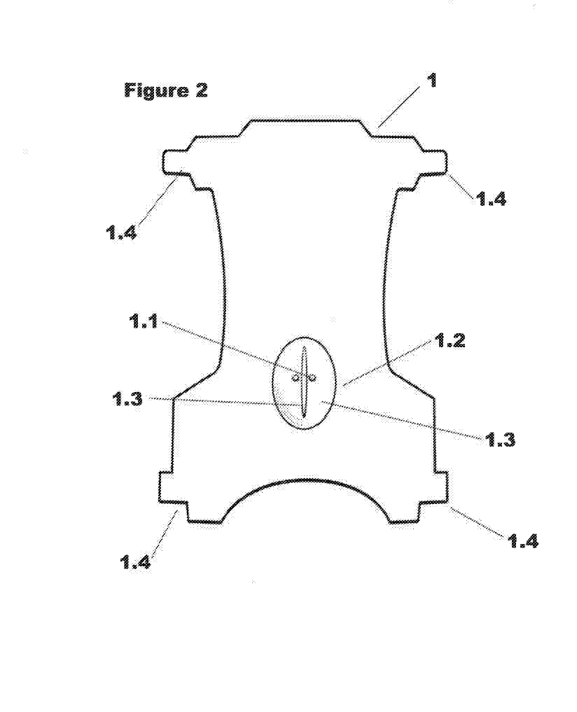

FIG. 2 shows the article of clothing 1 opened up to illustrate the foundation area 1.2. The foundation area can also be fitted with an opening 1.1 leading between the sheets to a pocket area where the urine collection device can be fitted. At least one or two openings 1.3 allow the connectors from the urine collection device to fit through.

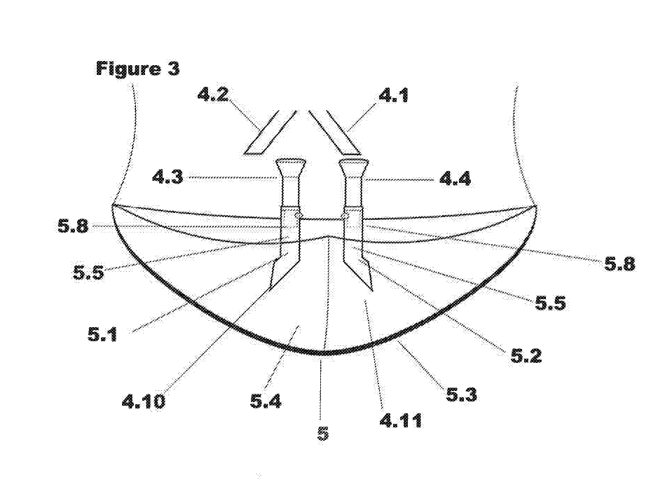

FIG. 3 shows urine collection bag 5 fitted with two connectors 5.1 and 5.2; the two connectors can be fitted with an air release valves 5.5 to aid the flow of urine. At least two flow valves 4.10 and 4.11 can be fitted to aid the flow of urine and transport the urine into the separate storage areas 5.4 and 5.3. Check valves 5.8 are fitted to the connectors to the lower end of the chambers 4.3 and 4.4 to prevent the urine from flowing back into the urine collection device. The drawings also illustrate the first catch funnel 4.1 engaged to transport urine into the first stream chamber 4.4 whist the mid-steam funnel 4.2 is disengaged from the mid-stream chamber 4.3.

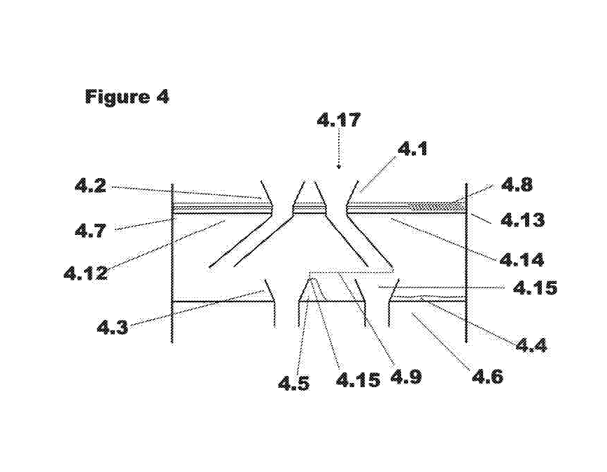

FIG. 4 shows the urine catch apparatus that is inside the urine collection device, the catch funnels 4.2 and 4.1 are seated in an encompassed surface 4.12 to become one item; and is held in place by the tension of a spring or other resilience component 4.8 that is fastened by a fixture or by catching, suspending, or connecting with a hook onto the inside wall 4.13 where is can be mounted; and the adjacent end of the spring or resilience component can be connected to the encompassed surface/catch funnels 4.14. A release strip 4.9 is also fastened to either the inside wall or the catch chamber as illustrated; and connected to the encompassed surface/catch funnels 4.3 by a fixture or by catching, suspending, or connecting with a hook. This will ensure that the device is ready to receive the first urine stream through the catch chamber 4.1 onto the release strip 4.9 which will weaken and disjoin after a period of time post the said first urine stream.

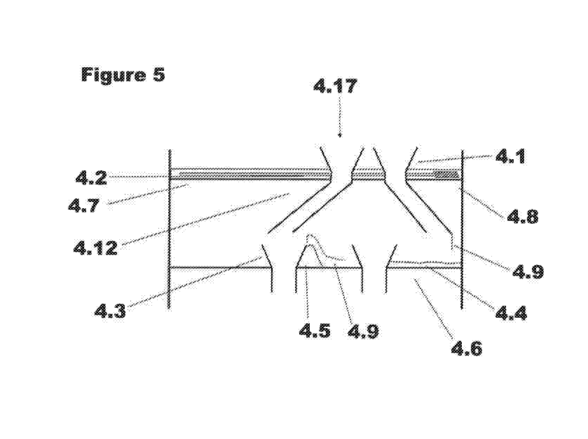

FIG. 5 shows the release strip 4.9 disjoined inside the apparatus that is inside the urine collection device, the encompassed surface/catch funnel is able to slide in between the support slides 4.7 to engage the mid-stream funnel 4.2 and mid-stream chamber 4.3 so that the mid-steam flow of urine can be collected for sampling. A fluid absorbent material 4.5 and 4.6 is fixed to a surface to absorb any excess urine.

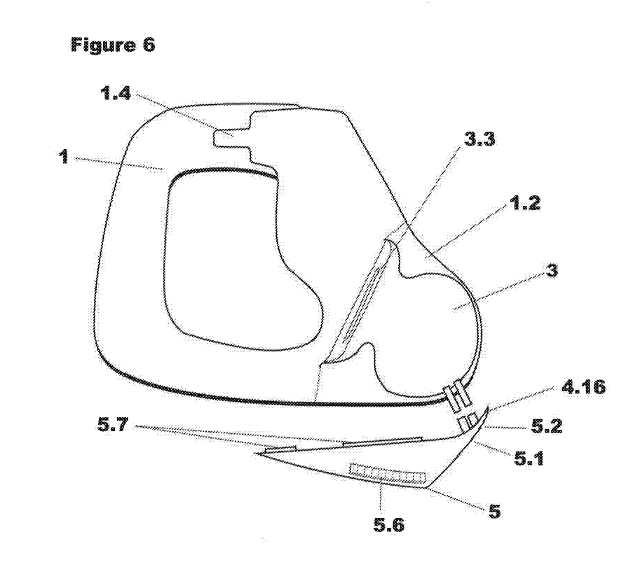

FIG. 6 shows a urine collection apparatus from a side view comprising the foundation area 1.2 and one of the arranged shapes of the article of clothing 1; and the urine collection device 3 inside the article of clothing 1; and the urine collection bag 5 which fits on the base; and the fastenings 5.7 to fix the urine collection bag to the base of the article of clothing ; and fastenings 1.4 to tighten the urine collection apparatus around the wearers waist; and the urine diagnostic strip 5.6 that can be fitted as part of the urine collection bag; and the opening 3.3 to receive the genital organ.

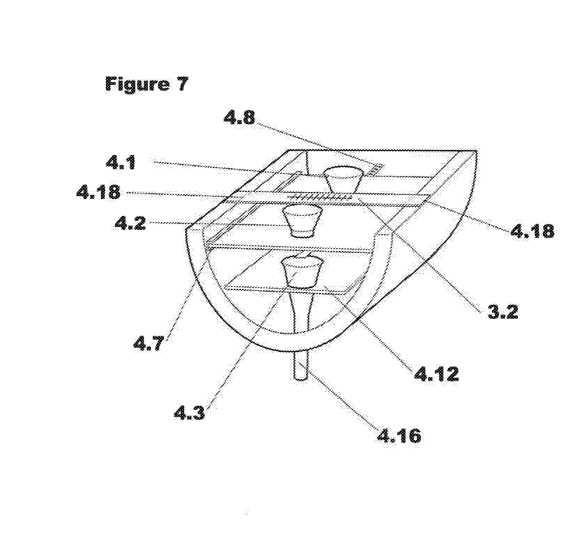

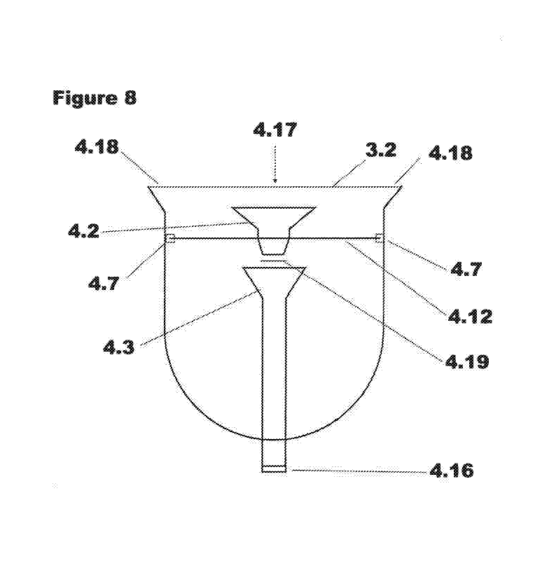

FIG. 7 and FIG. 8 show a urine collection device comprising the catch funnels and sliding supports 4.7 fixed to the inner wall of the urine collection device. The flexible support 3.2 is fixed to the opening of the urine collection device and is perforated 4.18 so it can be removed if needed. The flexible support is designed to support the penis and therefore can be removed when being used by a female.

Whilst the above description in relation to FIGS. 1 to 8 has focussed on the use of plural catch funnels (4.1 and 4.2 in the drawings) it is also possible to deploy a single catch funnel to embody the inventive concept of the invention.

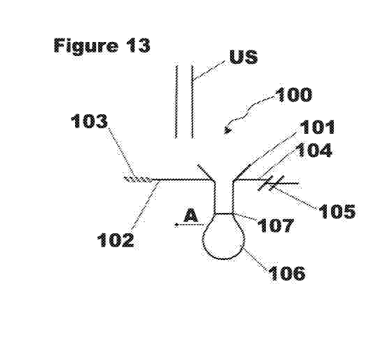

Referring to FIG. 13, there is a shown a schematic representation of a further catch device 100 according to the invention in which there is shown a catch device or funnel 101 biased in the direction of arrow A by a biasing member 102, for example, resilient member 103. The catch funnel 101 is restrained against the action of the biasing member 102 by a holding member 104.

Thus, in a first condition, as shown in FIG. 13, the catch funnel 101 is retained out of the intended flow path of a urine stream US. At least a portion of the urine may be collected, or may soak into, for example, surrounding material. In some embodiments surrounding material may be material of a nappy or other sanitary wear. At least some of the urine may pool in, or flow to, the proximity of a break portion 105 in the holding member 104. The break portion 105 is arranged to fracture, tear, or at least become weakened, on contact with urine.

As the break portion 105 fractures, tears or otherwise weakens the force of the biasing member 102 overcomes the holding force exerted by the holding member 104 thereby causing the catch funnel 101 to be urged into engagement with the urine stream US, whereby a mid-stream catch of urine will enter the catch funnel 101 and may be subsequently directed into a downstream chamber 106. In this way, the initial flow of a urine stream is discarded (e.g. being absorbed into surrounding material such as material of a nappy or other sanitary wear) and only the subsequent flow is captured by the catch funnel 101. This arrangement is the second condition of the device 100.

Thus it can be appreciated that the device allows the capture of just the desired mid-stream flow of urine.

The catch funnel 101 and/or the downstream chamber 106 may be provided with a non-return valve 107 or other flow inhibitor, thereby to prevent captured urine from leaking from the chamber 106.

The inherent strength of the break portion 105 is chosen according to the intended user (e.g. taking into account the velocity, volume, or other characteristic of the likely urine stream

US). For example, for an infant (baby or toddler) the strength may be low; for a large animal (e.g. a horse) the strength may be relatively high. Moreover, if it is required that a relatively large sample is taken the strength of the break portion may be chosen such that it weakens only when it has been exposed to a relatively large amount of urine, for example. Weakening of the break portion 105 may be carried out in any of the ways contemplated above.

The catch funnel 101 may extend through or be otherwise supported on a support member. The support member may be operatively connected to the biasing member 102 and/or the holding member 104. In that case, as the break member 105 fractures or weakens the resilience of the biasing member 102 acting on the support member will cause the catch funnel 101 to be brought into a catching relationship with the urine stream US, thereby to catch a portion of the urine stream US once at least a portion has been discharged from the urethra to waste (i.e. that initial portion has not entered the catch funnel 101, and thus the funnel is uncontaminated when it receives the mid-stream urine sample).

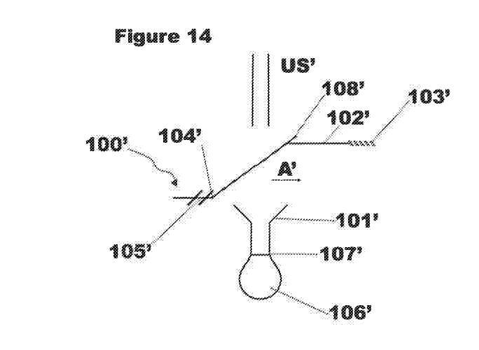

A further embodiment of the invention is shown in FIG. 14, which provides a schematic representation of a further of device 100′ according to the invention.

The device 100′ is provided with a catch device or funnel 101′ for collecting urine from a urine stream US′. The catch funnel 101′ communicates with a downstream collection chamber 106′ and one or both of the catch funnel 101′ and chamber 106′ may be provided with a non-return valve 107′ or other flow inhibitor, thereby to prevent captured urine from leaking from the chamber 106.

The device 100′ also comprises a baffle 108′ or other flow diverter which is biased in the direction of Arrow A′ by action of a biasing member 102′ which comprises a resilient member 103′. The action of the biasing member is restrained by a restraining member 104′ which is provided with a break member 105′.

Thus, in a first condition, as shown in FIG. 14, urine from the urine stream US′ is unable to pass to the catch funnel 101′ because the urine is diverted by the baffle 108′ away from the catch funnel 101′.

At least a portion of the urine may be collected, or may soak into, for example, surrounding material. In some embodiments surrounding material may be material of a nappy of other sanitary wear. At least some of the urine may pool in, or flow to, the proximity of a break portion 105′ in the holding member 104′. The break portion 105′ is arranged to fracture, tear, or at least become weakened, on contact with urine.

As the break portion 105′ fractures, tears or otherwise weakens the force of the biasing member 102′ overcomes the holding force exerted by the holding member 104′ thereby causing the baffle 108′ to be urged out of the way of the urine stream US′ and thereby enabling the catch funnel 101′ to communicate with the urine stream US′, whereby a mid-stream catch of urine will enter the catch funnel 101′ and may be subsequently directed into a downstream chamber 106′. In this way, the initial flow of a urine stream US′ is discarded (e.g. being absorbed into surrounding material such as material of a nappy or other sanitary wear) and only the subsequent flow is captured by the catch funnel 101′. This arrangement is the second condition of the device 100′.

Thus it can be appreciated that the device allows the capture of just the desired mid-stream flow of urine.

The inherent strength of the break portion 105′ is chosen according to the intended user (e.g. taking into account the velocity, volume, or other characteristic of the likely urine stream US). For example, for an infant (baby or toddler) the strength may be low; for a large animal (e.g. a horse) the strength may be relatively high. Moreover, if it is required that a relatively large sample is taken the strength of the break portion may be chosen such that it weakens only when it has been exposed to a relatively large amount of urine, for example. Weakening of the break portion 105′ may be carried out in any of the ways contemplated above.

The break portion 105′ may be provided on the baffle or other flow diverter 108′. If so, the holding member 104′ (which acts as a restraining portion) may also be resiliently urged, preferably in a direction opposite to that of arrow A′.

In both the embodiments discussed in relation to FIGS. 13 and 14, it is the movement of the catch funnel 101 or a baffle or other flow diverter 108′ which means the initial portion of the urine stream does not enter the catch funnel 101/101′ but the mid-stream sample does enter the catch funnel 101/101′.

The catch funnel 101/101′ can be made in accordance with any of the embodiments discussed in this specification, as can the chamber 106/106′. The catch funnel 101/101′ may not be tapered outwardly, for example it may have a constant cross section or inwardly tapering cross section towards the distal end. The catch funnel 101/101′ may also be protected by a urine dissolvable member (or other urine deflector or absorber) to reduce the likelihood of polluting splashes from entering the catch funnel before the desired mid-stream portion. Moreover, the device 100, 100′ can be provided with a further catch funnel, for example to catch the first portion of urine from the urine stream US/US′ and/or a subsequent portion of urine from the urine stream US/US′. For example, the break portion 104 may be provided in two parts, a first to allow limited movement of the catch funnel 101 into the second condition and then may break again (or a further portion may break) to bring a succeeding catch funnel into communication with the stream US.

All and each of the features discussed above in relation to the embodiments of FIGS. 1 to 8, or otherwise, can be incorporated or used in the devices 100, 100′ for collection of a mid-stream urine sample from a patient as shown in the FIGS. 13 and 14.

The restraining portion 104/ 104′ and/or break portion 105/105′ can be formed from, for example a urine-dissolvable material; for example the portion may comprise one or more of (a) cellulose-based material, such as paper or card, or (b) a polymer that dissolves or disintegrates in the presence of water, or (c) a polymer that dissolves or disintegrates at the pH of urine, or (d) material that includes a pre-weakened section, e.g. a perforated section, that will break under the downward force of urine flowing onto it.

The biasing member 102/102′ may be a spring or, for example a wish bone, or other urging member.

When the device is used with a male person, the person's penis is usually restrained, to ensure that the direction of the urine stream is known at least approximately.

The other embodiments discussed above and below provide details of how the device can be used and/or installed in, for example, clothing.

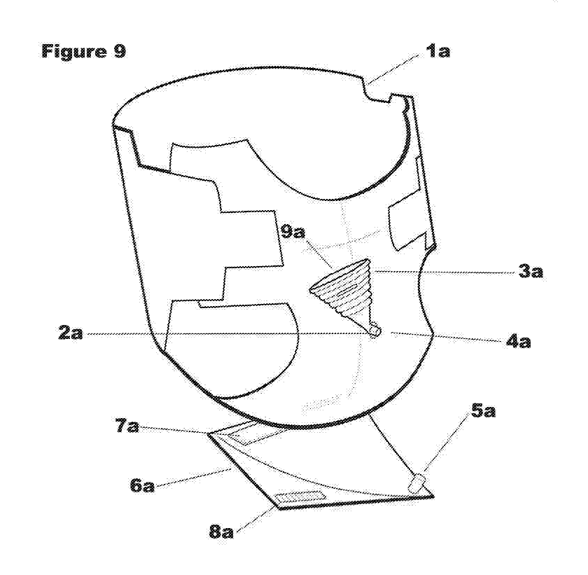

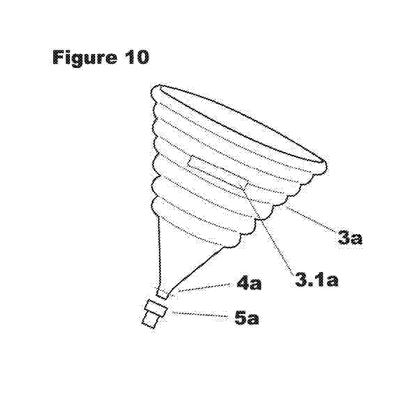

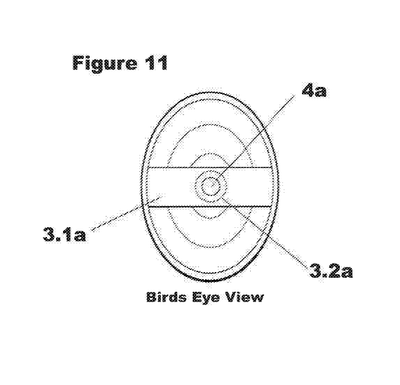

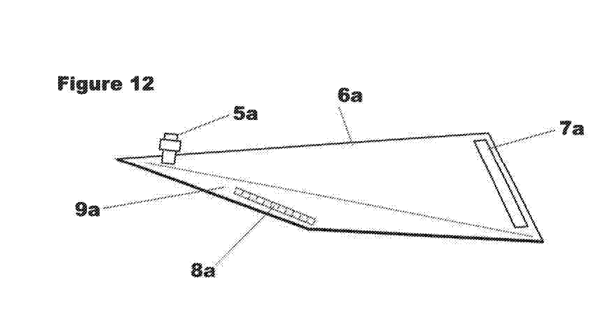

FIGS. 9-12 show another urine collection device of the present invention. This device is designed for the collection of a urine sample from a patient, whilst allowing the patient to carry on with their normal routine and activities.

The device includes a urine collection chamber 6a. The device also includes a catch funnel 3a for receipt of urine, the catch funnel having an entry portion for receiving a urine stream from the patient and an exit portion for transferring said urine stream to the urine collection chamber.

The device may include a garment 1a suitable for being worn by the patient around their lower torso and/or genital area. This may be a nappy, or underpants, an incontinence clothing article or the like. When the garment is worn by the patient around their lower torso and/or genital area, the urine collection chamber and the catch funnel are located inside the garment, adjacent to the patient's body, with the entry portion of the catch funnel being located proximate the patient's urethra, such that the urine collection device is in a configuration in which a sample of the patient's urine stream can be received in the entry portion of the catch funnel and transferred via the exit portion of the catch funnel to the urine collection chamber.

Alternatively, the device need not itself include the garment but may be designed for use with such a garment.

In the event that the urine collection chamber is not provided in a form where it is attached to a garment, the device will include a securing component 7a for securing the urine collection chamber to the patient's garment. This may be mechanical or chemical. For example it may involve the use of high friction material, which will grip the securing component to the fibres of the patient's garment such that the securing component can simply be pressed onto the patient's garment to secure the urine collection chamber to the inside of the garment, or may involve a chemical adhesive, like those known for use in nappies and sanitary pads, such that the securing component can simply be pressed onto the patient's garment to secure the urine collection chamber to the inside of the garment. It could be that the securing component comprises a hook type securing device, or hook and loop type securing device, or a flocked material.

The urine collection device is thus able to be provided in a configuration in which the securing component secures the urine collection chamber to the patient's garment, and the entry portion of the catch funnel is located proximate the patient's urethra, such that in this configuration a sample of the patient's urine stream can be received in the entry portion of the catch funnel and transferred via the exit portion of the catch funnel to the urine collection chamber.

FIGS. 9-12 also show a hole 2a in the garment 1a; this hole may be stitched around its edges and provides a gap through which a connector tube can pass. Also shown is a connector 4a that connects to the catch funnel and a connector 5a that connects to the urine collection chamber; these two connectors may therefore be joined together to connect the catch funnel and the urine collection chamber.

Also shown is the optionally included urine test strip 8a for testing the urine sample straight away. There is a flexible support 3.1a that holds the male's penis in the hole 3.2a to obtain a clean-catch urine sample avoiding any contamination.

The urine stream is marked as 9a.

Considering the drawings further, the following description can be provided:

The garment, which may be a nappy or other garment for urine sample collection, 1a is designed with a hole 2a which the urine collection component connector 4 fits through. The urine collection component 3a is a flexible item to allow comfort when fitted to the groin area around the genitals and is fitted with flexible support 3.1a that holds the male's penis in the hole 3.2a to obtain a clean-catch urine sample avoiding any contamination. Flexible support 3.1a can be removed for female patients. The urine collection component 3 is connected to the urine bag/container connector 6a by connecting the urine collection bag/container connector 5a to the urine collection component connector 4a. The fixture strip 7a is attached to the nappy 1a. The nappy 1 is fitted onto the patient. The urine collection component 3 is fitted around the groin area over the genitals to collect the urine. The urine passes through the urine collection component 4 and is collected into the urine collection bag/container 5a. When the urine makes contact with the urine test strip 8a which is fitted onto the bag/container. The urine test strip 8a changes colour according to the results within the urine 9a. The urine bag/container 6a is removed by disconnecting the urine bag/container 6a connector from the urine collection connector 4a. The urine bag/container can be taken to the lab for further analysis.

1-22. (canceled)

23. A device for collection of a mid-stream urine sample from a patient, the apparatus comprising:
a catch device for the receipt of urine from a urine stream;
a biasing component; and
a release component;
wherein the device has a first condition in which urine from a urine stream is unable to enter the catch device and a second condition in which urine from the urine stream is able to enter the catch device, said biasing component urging the apparatus towards the second condition and being operatively restrained by the release component; and wherein, with the device in the first condition, urine from a urine stream causes or is able to cause the release component to cease operatively restraining the biasing component whereby the device adopts the second condition under action of the biasing component; and wherein in the first configuration said biasing component operatively urges the catch device into or towards an intended flow path of a urine stream.

24. The device according to claim 23, comprising a baffle or other urine deflector arranged to intercept an intended flow path of a urine stream.

25. The device according to claim 24, wherein in the first configuration said biasing component operatively urges the baffle or other urine deflector out of engagement with an intended flow path of a urine stream and said release component operatively restrains said biasing component.

26. The device according to claim 23, comprising an initial catch device, said initial catch device being arranged to collect urine from a urine stream with said device in said first condition.

27. The device according to claim 23, comprising:
as an initial catch device, a first catch funnel for receipt of urine, the first catch funnel having an entry portion for receiving a urine stream and an exit portion for transferring said urine stream to a first urine collection chamber;
as the catch device, a second catch funnel for receipt of urine, the second catch funnel having an entry portion for receiving a urine stream and an exit portion for transferring said urine stream to a second urine collection chamber;
a support component on which the first catch funnel and second catch funnel are mounted;
wherein the biasing component biases the support component towards a position where the second catch funnel is located to receive the urine stream from the patient; and
wherein the release component releasably secures the support component against the bias of the biasing component, to be in a position where the first catch funnel is located to receive the urine stream;
wherein the urine collection device can be initially provided in the first configuration, in which the release component secures the support component against the bias of the biasing component, with the support component being in a position where the first catch funnel is located to receive the urine stream from the patient, and in this first configuration a first sample of the urine stream can be received in the entry portion of the first catch funnel and transferred via the exit portion of the first catch funnel to a first urine collection chamber,
and wherein the urine collection device is able to subsequently be provided in the second configuration, in which the release component no longer secures the support component against the bias of the biasing component, such that the biasing component acts to move the support component to a position where the second catch funnel is located to receive the urine stream from the patient, and in this second configuration a mid-stream sample of the urine stream can be received in the entry portion of the second catch funnel and transferred via the exit portion of the second catch funnel to a second urine collection chamber.

28. The device according to claim 27, wherein the support component slides between a first position, where the first catch funnel is located to receive the urine stream from the patient, and a second position, where the second catch funnel is located to receive the urine stream from the patient.

29. The device according to claim 23, wherein the biasing component is a resilient or elasticated component.

30. The device according to claim 23, wherein the biasing component is a spring.

31. The device according to claim 23, wherein the release component comprises a break portion that is weakened by contact with urine.

32. The device according to claim 31, wherein:
(a) the contact of the urine stream on the break portion causes the break portion to weaken and break, thereby releasing the support component or removing restraint of the biasing component; or
(b) the contact of the urine stream on the break portion causes the break portion to weaken and the combination of this weakening and the biasing force from the biasing component causes the break portion to break, thereby releasing the support component or removing restraint of the biasing component.

33. The device according to claim 31, wherein the break portion:
(a) is made from cellulose-based material, such as paper or card, or
(b) is made from a polymer that dissolves or disintegrates in the presence of water, or
(c) is made from a polymer that dissolves or disintegrates at the pH of urine, or
(d) includes a pre-weakened section, e.g. a perforated section, that will break under the downward force of urine flowing onto it.

34. A device according to claim 23, wherein the catch device comprises a non-return means for securing a sample of urine collected within the device.

35. A garment for wearing by a human or non-human animal incorporating a device according to claim 23, which device is located on or in the garment for the collection of a urine sample from the wearer.

36. The garment according to claim 35, wherein the garment is a nappy, underpants, an incontinence clothing article, a belt that can strap around the lower torso, or a pair of shorts or trunks.

37. A device for collection of a mid-stream urine sample from a patient, the apparatus comprising:
a catch device for the receipt of urine from a urine stream;
a biasing component; and
a release component;
wherein the device has a first condition in which urine from a urine stream is unable to enter the catch device and a second condition in which urine from the urine stream is able to enter the catch device, said biasing component urging the apparatus towards the second condition and being operatively restrained by the release component;
and wherein, with the device in the first condition, urine from a urine stream causes or is able to cause the release component to cease operatively restraining the biasing component whereby the device adopts the second condition under action of the biasing component and wherein the biasing component is a spring.

38. A device for collection of a mid-stream urine sample from a patient, the apparatus comprising:
a catch device for the receipt of urine from a urine stream;
a biasing component; and
a release component;
wherein the device has a first condition in which urine from a urine stream is unable to enter the catch device and a second condition in which urine from the urine stream is able to enter the catch device, said biasing component urging the apparatus towards the second condition and being operatively restrained by the release component;
and wherein, with the device in the first condition, urine from a urine stream causes or is able to cause the release component to cease operatively restraining the biasing component whereby the device adopts the second condition under action of the biasing component and wherein the catch device comprises a non-return means for securing a sample of urine collected within the device.
